(19) United States
(12) Patent Application Publication
(10) Pub. No.: US 2026/0000297 A1
(43) Pub. Date: Jan. 1, 2026

(54) METHOD AND SYSTEM OF MEDICAL MULTI-DYE FLUORESCENCE IMAGING

(71) Applicant: OLYMPUS Winter & Ibe GmbH, Hamburg (DE)

(72) Inventors: Konrad ANDERS, Hamburg (DE); Mario BOCK, Hamburg (DE)

(73) Assignee: OLYMPUS Winter & Ibe GmbH, Hamburg (DE)

(21) Appl. No.: 19/181,783

(22) Filed: Apr. 17, 2025

Related U.S. Application Data
(60) Provisional application No. 63/665,770, filed on Jun. 28, 2024.

(51) Int. Cl. A61B 5/00; G06T 5/50; G06T 5/60; G06T 5/70; G06T 7/00; G06T 7/90; G06T 11/00

(52) U.S. Cl. CPC A61B 5/0071; G06T 5/50; G06T 5/60; G06T 5/70; G06T 7/0016; G06T 7/90; G06T 11/001; G06T 2207/10016; G06T 2207/10024; G06T 2207/10064; G06T 2207/10152; G06T 2207/20081; G06T 2207/20216; G06T 2207/30028

(57) ABSTRACT

A method of medical multi-dye fluorescence imaging. The method including: capturing fluorescent images of an operating field in which two or more different fluorescent dyes are present in successive sequences of images with a surgical or diagnostic imaging device, each sequence of images alternating through different modes of excitation lighting adapted for at least one each of two or more of the two or more different dyes, and processing the captured images of each successive sequence of images in combination with each other. The processing comprising determining the respective local distributions of the two or more different fluorescence dyes causing the observed different distributions of brightness in the two or more different fluorescent images of the sequence and producing one or more images displaying the determined local distributions of the two or more different fluorescent dyes separately.

### CROSS-REFERENCE TO RELATED APPLICATION

The present application is based upon and claims the benefit to U.S. Provisional Patent Application No. 63/665,770 filed on Jun. 28, 2024, the entire contents of which are incorporated herein by reference.

### BACKGROUND

### Field

The present disclosure relates to a method and a system of medical multi-dye fluorescence imaging.

### Prior Art

Medical applications of fluorescence imaging, a subtype of molecular imaging, are fluorescent image guided surgery and diagnostics, in which a medical imaging technique used to detect fluorescent substances with the purpose of guiding the surgical or diagnostic procedure and providing the operator with real time visualization of the operating field, both in open surgery and in endoscopic and robotic procedures, as well as in diagnostic procedures. Fluorescence dyes (fluorophores) commonly used for different applications in fluorescent image guided surgery and diagnostics include indocyanine green (ICG) and others in the optical and near infrared spectrum, but can also include dyes that fluoresce at other wavelengths, such as the ultraviolet or the far infrared spectrum or any wavelengths that can be processed by an image processor.

Fluorescence imaging is a form of molecular imaging, which generally encompasses imaging methods for visualizing and/or tracking of molecules having specific properties that are used for molecular imaging. Such molecules can be substances that are endogenous to the body, or dyes such as targeted dyes, i.e., antibody or enzyme labeled tracers that accumulate in structures, i.e., body cells of interest, or contrast agents that are injected into the patient. MRI and CT, for example, therefore, also fall under the term “molecular imaging”. Fluorescence imaging as a variant of molecular imaging uses the property of certain molecules (fluorophores), which emit light of certain wavelengths when excited by light of certain excitation wavelengths.

For the purpose of fluorescence imaging, the system's imaging system, e.g., a camera head, typically includes sensors that are sensitive in the visible spectrum and in the near infrared spectrum, but may also cover other spectra, depending on the dyes used. The system's illumination light source unit has a light source for white light to illuminate the operating area with white light as well as at least one excitation light source designed for exciting at least one fluorescent dye present in the operating area. The excitation light source may comprise a laser or a light emitting diode, the wavelength chosen to excite fluorescence in the dye being used. After being excited, the dyes shed the excitation energy by emitting light at slightly longer wavelengths than that of the excitation light. This emitted light passes through a high pass filter and is then captured by an image sensor. Other wavelengths may be used as excitation wavelengths depending on the type of dye used. This can include wavelengths that are further inside the visible spectrum or further outside the visible or infrared spectrum.

Based on the foregoing, the image processing system generates a composite image with an overlay of the fluorescent image over a non-fluorescent image for easy localization of the fluorescent areas within its surroundings and displays it to the surgeon on a screen. When overlaid over the white light images, the fluorescent images are usually converted into a false color image, for example in a light green color in the case of ICG as a dye, that ideally contrasts the red hues of the white light image, with the brightness indicating the intensity of the fluorescent emission.

Various dyes with different properties including different fluorescence excitation and emission spectra are available to highlight different structures during surgery, like the usage for lung or colon, or for highlighting tumors and critical structures such as ureter, lymphatics vessels and nerves.

In some use cases, it may be beneficial to use two or more different fluorescent dyes for different purposes simultaneously. An exemplary such use case is the colectomy procedure, where surgeons would like to visualize within one procedure the fluorescence dyes SGM-101 (MI700) for colorectal cancer observation as well as ICG (MI800) for blood flow observation. Both fluorescent dyes are injected into the patient.

These two fluorescent dyes have different excitation and emission spectra, with emission spectra being narrow, non-overlapping, and centered around 700 nm and 800 nm, respectively. The excitation spectrum of these two fluorescent dyes, however, do overlap. The excitation of ICG may be performed using, for example, a laser at a wavelength of 785 nm. This wavelength is inside the excitation spectrum of ICG, and outside of the excitation spectrum of SGM-101. The excitation of SGM-101, however, may be typically performed using, for example, a laser at a wavelength of 685 nm. This latter wavelength lies within the excitation spectra of both SGM-101 and ICG, resulting in crosstalk, with ICG being excited alongside with SGM-101.

The technical challenge posed by this crosstalk of exciting more than one fluorescent dye with a single excitation wavelength is common to all combinations of different fluorescent dyes having overlapping excitation spectra. It is solved by using imaging systems having three or more image sensors of which one is sensitive to white light and provides white light imaging, whereas the other ones are sensitive in the ranges, be it visual or infrared, wherein the chosen fluorescent dyes emit their fluorescence light. These two or more image sensors are each dedicated exclusively to detecting the fluorescence light of a single one of the two or more fluorescent dyes by having directly in front of the respective sensor a narrowband filter (also called a wavelength cut-filter) transmitting only light of the emission wavelength of the respective fluorescent dye. The fluorescent emission of the other dyes is blocked out, so that there is a clean signal for each of the injected fluorescent dyes.

Another solution is needed for the case where the imaging system only has two image sensors, namely a first image sensor for the visual spectrum and a second image sensor sensitive in the infrared spectrum, frequently with some overlap into the red part of the visible spectrum. Neither of the two image sensors in that case is equipped with a narrowband filter. Hence, the second image sensor will receive and be sensitive to any fluorescence light emitted by any fluorescent dye injected into the patient and excited by the currently used excitation light.

### SUMMARY

Based on this, an object of the present disclosure is to provide a method and a system for molecular imaging with two or more fluorescent dyes, when there is no filtering involved in the image capturing.

Such object can be solved by a method of medical multi-dye fluorescence imaging comprising: capturing fluorescent images of an operating field in which two or more different fluorescent dyes are present in successive sequences of images with a surgical or diagnostic imaging device, each sequence of images alternating through different modes of excitation lighting adapted for at least one each of two or more of the two or more different dyes, processing the captured images of each successive sequence of images in combination with each other, the processing comprising determining the respective local distributions of the two or more different fluorescence dyes causing the observed different distributions of brightness in the two or more different fluorescent images of the sequence and producing one or more images displaying the determined local distributions of the two or more different fluorescent dyes separately.

The method relies on capturing images of the operating area under separate lighting modes for the various fluorescent dyes and subsequently processing those images to gather such as to disentangle the contributions of the various fluorescent dyes under the various different excitation lighting modes. The result is the information about the distribution of the different fluorescent dyes in the operating area without the need to apply an equal number of image sensors with filters making them sensitive for a specific fluorescent dye.

The proposed mode of processing makes use of the physics underlying the excitation of fluorescent dyes. Each fluorescent dye emits fluorescence light in its own narrow spectral range when excited. Furthermore, each fluorescent dye can be excited to fluoresce by being exposed to excitation lighting, wherein the excitation light has more energy, equivalent to a shorter wavelength, than the fluorescence emission light of that fluorescence dye. Each fluorescence dye is sensitive to excitation light in a somewhat broader excitation spectral range according to a sensitivity distribution, wherein peak sensitivity for excitation is usually achieved at the longer wavelengths range of the respective excitation spectrum of the individual fluorescence dye, and falling of steeply towards shorter wavelengths.

Because of the high peak sensitivity of the fluorescent dye in the vicinity of the fluorescence emission peak, fluorescence excitation light is usually produced with a shift of 10 to 25 nm in the direction of shorter wavelengths with respect to the peak wavelength of the fluorescence emission. Also, whereas the fluorescence emission spectra of typically used fluorescent dyes have a width of a few 10 nm, with a tail extending towards greater wavelengths, i.e., into the infrared, the excitation spectra, that is the spectral range is in which they are sensitive to excitation light, can extend up to 100 nm or more towards shorter wavelengths with respect to the respective fluorescence emission peak.

In cases that different fluorescent dyes are used as markers for different structures or tissue types, the fluorescence emission spectra of the different dyes usually do not overlap. This is used in multi-sensor applications by using one image sensor for each fluorescent dye with a filter that allows only the fluorescence emissions from one specific dye to pass, whereas all other wavelengths, including the excitation wavelength, are blocked from reaching the image sensor. In this way, a very clean signal is reached at the cost of a complicated and involved instrumentation.

However, in case that only one image sensor is used to detect fluorescence images from the various dyes used in the operating area, the image sensor may not be able to discriminate between different wavelengths and will only record the intensity of fluorescence light being captured from the operating area. Filtering may only be applied to block out excitation illumination for the various dyes by using narrowband filters tuned to the excitation wavelengths used. In this case, the excitation spectra of the various fluorescent dyes will likely overlap to some extent, so that fluorescence excitation illumination be tuned to a specific fluorescent dye through the choice of wavelength in the peak of the sensitivity distribution of that fluorescent dye will also be inside the short wavelength tail of the sensitivity distribution of another fluorescent dye having a fluorescence emission peak at a longer wavelength than the first fluorescence dye. However, the fact that the second fluorescent dye is still sensitive for excitation at that shorter wavelength results in the second fluorescent dye being excited along with the first fluorescence dye, albeit to a lesser extent.

The strength of the fluorescence response to excitation light of each fluorescent dye tends strongly on the wavelength of the excitation light, which means that by capturing subsequent images of fluorescence emissions from two or more different fluorescent dyes in an operating area, the subsequent images being produced with alternating fluorescence lighting attuned to a first and a second, and possibly a third and so forth, fluorescent dye, it is possible to analyze the different images in combination with each other and to extract the distributions of the individual fluorescence dyes therefrom.

Excitation lighting modes that provide excitation lighting in the wavelength ranges of peak sensitivity of the individual fluorescent dyes can be used, since such choice will provide maximum contrast in most cases. In other cases, especially, if more than two different fluorescent dyes are used, excitation lighting modes in wavelength steps that are not particularly tuned to the peak sensitivities of the different dyes can be used, but instead at predetermined wavelengths, that may be spaced apart by given step widths, such as 50 nm or 100 nm or the like. Such an infrastructure can be especially beneficial if the range of different fluorescent dyes is to be expanded over time.

In an embodiment, the processing can further comprise inputting the fluorescent images of the sequence of images into at least one artificial intelligence model trained to perform the determination of the respective local distributions of the two or more different fluorescence dyes, or into several artificial intelligence models that have each been trained on at least one of several different dyes. Such artificial intelligence models may be machine learning models adapted for image analysis, such as neural networks, for example, convolutional neural networks, that have been trained on training images of operating areas with fluorescent dyes present, under various fluorescence lighting modes. Such training images may comprise images that have only one specific and identified or labeled fluorescent dye present so that the machine learning model or artificial intelligence model may learn the excitation response of the individual fluorescent dyes along with other images that have two or more fluorescent dyes present. As the artificial intelligence model learns from the images directly, it is not necessary to have a priori knowledge of the individual responses of the different fluorescent dyes in use to the various excitation light wavelengths used.

In an embodiment, the processing may further comprise using linear combinations of the two or more fluorescent images with regard to their respective brightness distributions, the linear combinations being done with weighting factors determined from known or measured relative fluorescence response strengths of the two or more different fluorescent dyes at the two or more different modes of excitation, the weighting factors chosen such as to separate the responses of the two or more fluorescent dyes from each other.

A linear combination of two fluorescent images may comprise subtracting the brightness values of one image from the brightness values of the other image according to multiplicative factors chosen to eliminate the contribution of one of the fluorescent dyes from the resulting linear combination.

By way of example, under a first fluorescence excitation lighting mode, the fluorescence response of a first fluorescent dye may be known to be 100% and the fluorescence response of a second fluorescent dye may be known to be 0%, because the wavelength of the first fluorescence excitation lighting mode may be longer than the fluorescence emission wavelength of the second fluorescent dye. A first fluorescence image captured under the first fluorescence excitation lighting mode will therefore be a clean representation of the distribution of the first fluorescence dye inside the operating area with no contamination from a fluorescence response from the second fluorescent dye.

However, under a second fluorescence excitation lighting mode that is attuned to the second fluorescent dye, the second fluorescence excitation lighting mode having a shorter wavelength than the first fluorescence excitation lighting mode, the fluorescence response of the second fluorescent dye may be known to be 100% and the fluorescence response of the first fluorescent dye may be known to be 20%. A second image taken under this second fluorescence excitation lighting mode will therefore have a strong fluorescence signal from the second fluorescent dye, but with a contamination from the first fluorescent dye. However, since the respective strengths of the fluorescence responses of the different dyes to the different excitation wavelengths are known, it is possible to formulate two linear equations and a solution.

In this case, the brightness distribution of the first image will provide a clean indication of the distribution of the first fluorescent dye in the operating area. A composite image, in which the brightness distribution of the first image is subtracted with a multiplicative factor of 0.2 from the second image, will provide an indication of the distribution of the second fluorescent dye in the operating area. This factor of 0.2 assumes that other instrumental effects such as sensitivity dependence on wavelength, respective strengths of the different fluorescence excitation light sources, or differences in the distribution of intensity of the respective fluorescence illumination over the operating area from different light sources, have been properly eliminated by calibration.

However, the application of linear combinations of the various images of a sequence of alternating fluorescence excitation illumination modes this is warranted because the images have been captured in close proximity in time to each other.

The above-described example is also an embodiment, wherein in each linear combination, the contribution of a specific one of the two or more fluorescent dyes is left, whereas the contributions of the other one or more fluorescent dye or dyes can be eliminated. Typically, the contribution of a fluorescent dye is left untouched if its excitation spectrum is removed from the excitation spectra of the other fluorescent dyes, so that there is no or negligible overlap.

In various embodiments, each of the different modes of excitation lighting can comprise activating a different set of one or more fluorescence excitation lighting sources that are each adapted to produce fluorescence excitation lighting for a different one of the two or more different dyes. The simplest embodiment will entail activating each of the fluorescence excitation lighting sources individually and in sequence. However, in other embodiments, the sequences may contain different combinations of fluorescence excitation lighting sources that each excite a different set of fluorescent dyes. In some embodiments, at least one of the fluorescence excitation lighting sources is activated or is kept activated continuously, which means that it is present in each of the different excitation lighting modes.

In embodiments, the method can further comprise in each sequence of images additionally capturing at least one white light image under white light illumination, and producing a composite image of the at least one white light image with an overlay of the determined distribution or distributions of at least one of the two or more different fluorescent dyes. The white light illumination may also be activated or kept activated continuously, in case it is directed towards a dedicated imager and the fluorescence excitation is outside the visible light.

The overlay may in embodiments be performed using a false color representation having a different color for each of the different fluorescent dyes. The foregoing has been described as successive sequences of alternating lighting modes, which may or may not include white light illumination. Those alternating lighting modes may occur in a repetitive pattern, each repetition marking a closed sequence. However, the sequences may also have varying patterns, depending on various circumstances. For example, it might be desirable to only produce white light imaging images for a while when the overlay with fluorescence distributions is not needed. Also, in case that the disentanglement of the contributions from the various fluorescent dyes takes up too much time or computing resources, it may be contemplated to produce sequences of fluorescence illumination lighting for only every second or third, and so on, white light image, wherein the resulting disentangled representations of the different fluorescent dyes present are used to be overlaid over several, such as two or more, subsequent white light images. In case that the subsequent white light images picture a shift because of a movement of an endoscope or the like, the movement may be analyzed and applied to the representations of the brightness distributions for the separate fluorescent dyes as well.

In some cases, the fluorescence response of a certain of the two or more fluorescent dyes under the fluorescence lighting mode attuned to that fluorescent dye may be very weak and therefore contain an elevated level of noise. When using such noisy fluorescence response the fluorescence response images for other fluorescent dyes, the noise can be amplified and render of those other fluorescence images noisy, too. In order to avoid introducing more noise, the intensity of the fluorescence excitation light source for the fluorescent dye having the weak response can be increased, or to add another one, two or more activations of the respective fluorescence excitation light source to the recurring sequence of light source activations. When the multiple fluorescence images of the sequence that were captured under the same fluorescence excitation lighting mode are combined or averaged, the resulting fluorescence image has less noise than the first, single one, alone.

The signal-to-noise ratio (SNR) of an image is a known and basic metric of image quality. The above-described additional steps for reducing SNR may be employed when an automatic calculation of SNR for specific lighting modes reveals that the SNR exceeds a preset threshold. On the other hand, the number of repetitions of a single excitation lighting mode in a sequence may be limited to a preset maximum number.

Such object of the present disclosure can be also solved by a system for medical multi-dye fluorescence imaging comprising a controller comprising hardware, a plurality of light sources controlled by the controller, the plurality of light sources comprising two or more fluorescence excitation illumination light sources configured to provide excitation illumination, wherein each of the two or more fluorescence excitation illumination light sources is configured to generate a different mode of excitation lighting adapted to a different one of two or more different pre-selected fluorescence dyes, at least one imaging unit comprising a first image sensor sensitive for fluorescence light from the two or more different pre-selected fluorescence dyes, the controller being configured to: control the plurality of fluorescence excitation illumination light sources by activating the two or more different fluorescence excitation illumination light sources in successive sequences of activations, each sequence of activations alternating through different modes of excitation lighting adapted for at least one each of two or more of the two or more different dyes, receive first image data from the first image sensor in synchronization with the successive sequences of activations of the two or more different fluorescence excitation illumination light sources, process the first image data of each successive sequence of images in combination with each other, the processing comprising determining the respective local distributions of the two or more different fluorescence dyes causing the observed different distributions of brightness in the two or more different fluorescent images of the sequence and produce one or more images displaying the determined local distributions of the two or more different fluorescent dyes separately.

The system embodies the structural features needed to implement the above-described method of the present disclosure and provides a simple and low-cost embodiment for multi-dye fluorescence imaging.

The fluorescence excitation light sources may comprise laser diodes, light emitting diodes or bright white light sources having narrowband filters or other appropriate sources.

In embodiments, the plurality of light sources can further comprise a white illumination light source configured to provide white light illumination, the imaging unit can further comprise a second image sensor sensitive in a visible light spectrum, wherein the controller is furthermore configured to: control the white illumination light source by activating the white illumination light source such as to, within each successive sequence of activations, alternating through different modes of excitation lighting as well as white light illumination, receive second image data from the second image sensor in synchronization with the successive activations of the white illumination light source, and produce a composite image by overlaying the one or more images displaying the determined local distributions of the two or more different fluorescent dyes over a white light illumination image derived from the second image data.

The controller hardware can be, in embodiments, configured to run at least one artificial intelligence model trained to perform the determination of the respective local distributions of the two or more different fluorescence dyes, or into several artificial intelligence models that have each been trained on at least one of several different dyes, to which the fluorescent images of the sequence of images are input.

In further embodiments, the controller hardware can be configured to use linear combinations of the two or more fluorescent images with regard to their respective brightness distributions, the linear combinations being done with weighting factors determined from known or measured relative fluorescence response strengths of the two or more different fluorescent dyes at the two or more different modes of excitation, the weighting factors chosen such as to separate the responses of the two or more fluorescent dyes from each other.

Such object of the present disclosure can be further solved by a non-volatile data storage medium containing instructions for a computer that are configured for causing the computer to perform the method according to one of the above-described embodiments.

Further features will become evident from the description of embodiments, together with the claims and the appended drawings. Embodiments can fulfill individual features or a combination of several features.

### BRIEF DESCRIPTION OF THE DRAWINGS

The embodiments described below, without restricting the general intent of the invention, based on exemplary embodiments, wherein reference is made expressly to the drawings with regard to the disclosure of all details that are not explained in greater detail in the text.

In the drawings:

FIG. 1 illustrates excitation and emission spectra of ICG and SGM-101 fluorescent dyes,

FIG. 2 illustrates an embodiment of a system for multi-dye fluorescence imaging and

FIG. 3 illustrates an embodiment of a method for multi-dye fluorescence imaging.

In the drawings, the same or similar types of elements or respectively corresponding parts are provided with the same reference numbers in order to prevent the item from needing to be reintroduced.

### DETAILED DESCRIPTION

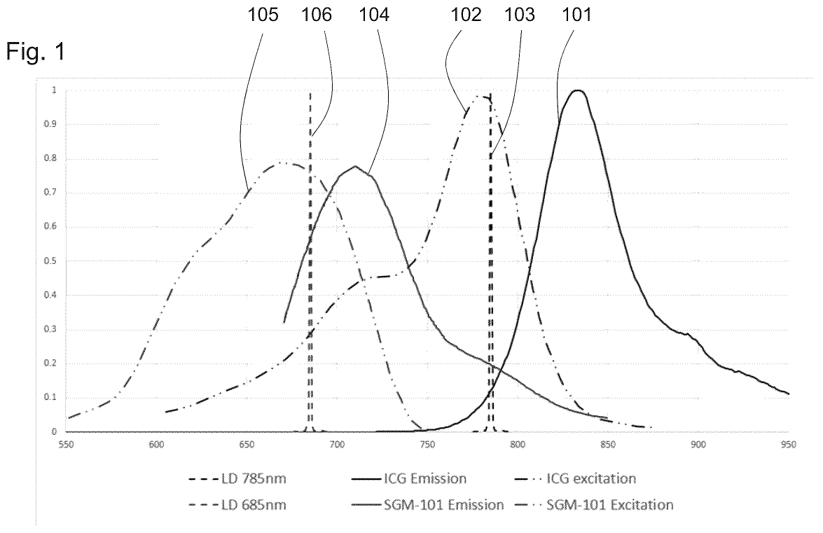

FIG. 1 illustrates excitation and emission spectra of indo cyanine green (ICG) and SGM-101 fluorescent dyes. The emission spectrum 101 of ICG is shown as a solid curve with a peak at approximately 817 nm and a tail extending into the infrared spectrum. Its excitation spectrum 102 is shown as dash-dotted line having a peak around 780 nm and a two extending into the visible spectrum up to 600 nm and more. The extreme tail end is not shown. Also shown is the very narrow excitation spectrum 103 of a laser diode tuned to 785 nm, which is attuned to the peak of the ICG excitation spectrum 102 and is therefore ideally suited to excite fluorescence emission from ICG.

Also shown in FIG. 1 are the emission spectrum 104 and the excitation spectrum 105 of SGM-101 fluorescent dye, with the peak of the emission spectrum 104 around 710 nm and having a tail extending towards longer wavelengths up to and exceeding 850 nm, whereas the excitation spectrum 105 defining the sensitivity of SGM-101 fluorescent dye peaks around 670 nm and extends towards shorter wavelengths up to and exceeding 550 nm. Also shown is the very narrow excitation spectrum 106 of the laser diode tuned to 685 nm, very close to the maximum sensitivity of SGM-101.

FIG. 1 shows clearly that the various spectra overlap significantly. For example, the infrared tail of the SGM-101-fluorescence emission spectrum 104 reaches into the ICG-fluorescence emission spectrum 101. More pertinent to the present disclosure, the excitation spectrum 102 of ICG and the excitation spectrum 105 SGM-101 overlap in the range from 600 nm to 750 nm.

This also means that the laser excitation spectrum 103 of the laser diode tuned to 785 nm lies outside the excitation spectrum 105 of SGM-101 and will only excite fluorescence emissions from ICG, but not from SGM-101. On the other hand, the laser excitation spectrum 106 of the laser diode tuned to 685 nm lies within the excitation spectra 102 and 105 of both SGM-101 and ICG, meaning that it will excite fluorescence responses from both fluorescent dyes. However, when exposed to 685 nm fluorescence excitation lighting, the response from SGM-101 will be 2 to 3 times stronger than the response from ICG. Since the relative strengths of the responses of ICG and SGM-101 for excitation light having a wavelength of 685 nm are known, it is possible to contribution of ICG by subtracting the fluorescence image brightness distributions taken at 785 nm, which is exclusively caused by ICG, from the image taken at 685 nm with an appropriate multiplicative factor.

The choice of ICG and SGM-101 as fluorescent dyes is by way of example and can be generalized to encompass several different fluorescent dyes having different excitation and emission spectra, some of which may overlap, usually in the visual and infrared spectra.

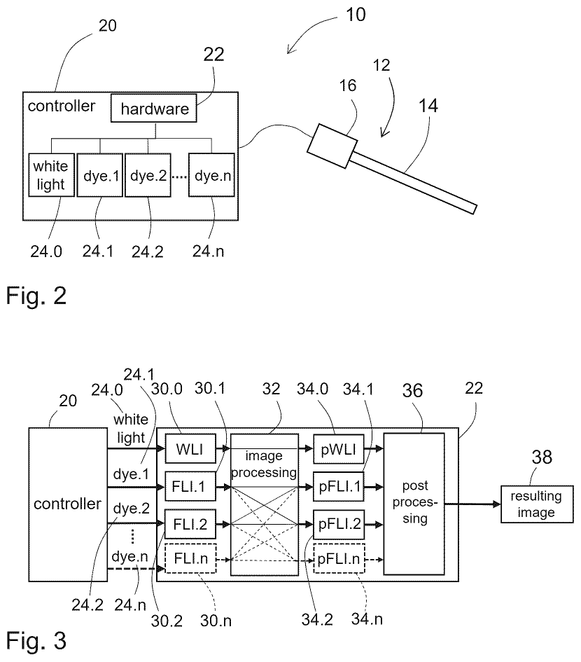

FIG. 2 illustrates an embodiment of a system 10 for multi-dye fluorescence imaging. The system 10 comprises an imaging unit such as an endoscope 12 having an endoscope shaft 14 and a camera head 16 having two or more sensors for white light imaging and for fluorescence imaging, respectively. Instead of an endoscope, a camera or an exoscope or the like may be used.

The system 10 further comprises a controller 20 having hardware 22, such as a central processing unit and components, as well as multiple light sources 24.0, 24.1, 24.2, . . . , 24.n controlled by controller 20. Light source 24.0, labelled “white light” in FIG. 2, may be a white light imaging light source generating white light, or a three color (RGB) light source having tuned lasers or LEDs. Light sources 24.1 through 24.n, labelled “dye.1” through “dye.n” in FIG. 2, are fluorescence excitation light sources, such as laser diodes, narrowband LEDs or bright white light sources with narrowband filters that let pass fluorescence excitation light configured to excite different dyes “dye. 1” through “dye.n”. Controlling hardware 22 is configured to activate light sources 24.0 through 24.n one at a time in sequences that may repeat themselves, and to capture images from the imaging unit that are taken under the lighting modes provided by the various light sources 24.0 through 24.n.

Controller 20, respectively controlling hardware 22, is configured to perform a method, an embodiment of which is symbolically illustrated in FIG. 3. Progressing from left to right, controller 20 causes the light sources 24.0 through 24.n to be activated in a sequence to successively, one at a time, provide illumination light for white light imaging and fluorescence excitation light for a first fluorescent dye (dye.1), a second fluorescent dye (dye.2), and so on, until an n^{th} fluorescent dye (dye.n), thus capturing a series of images 30.0, 30.1, 30.2, . . . 30.n of an operating area, with image 30.0 being a white light image labelled “WLI” in FIG. 3 captured by a white light color sensitive image sensor of the imaging unit and images 30.1 through 30.n being fluorescent images, labelled “FLI.1” through “FLI.n” in FIG. 3, taken under 1^{st}, 2^{nd}, . . . , n^{th} fluorescence lighting modes using a second image sensor that is sensitive in the spectral region of the fluorescence responses of the various fluorescent dyes present in the operating area. The minimum number of fluorescence lighting modes in the context of the present disclosure is two. The minimum number of white light images 30.0 per sequence is zero. However, there may be one, two or more white light images 30.0 captured before a sequence of fluorescence excitation lighting activations is performed.

In this embodiment, each of the lighting modes entails activating a single one of the fluorescence excitation light sources 24.1 through 24.n, or the white light illumination light source 24.0. In different embodiments, one or more of the lighting modes may entail activating different combinations of light sources 24.0 through 24.n, provided that no two lighting modes contain the same light sources in the same intensity ratio, which would render them unfit for the purpose of discriminating between the contributions of the different fluorescent dyes. This may entail keeping the white light illumination light source 24.0 and/or one or more of the fluorescence excitation light sources 24.1 through 24.n activated.

For example, in the simple case that two dyes are used, for example, SGM-101 (MI700) for colorectal cancer observation as well as ICG (MI800) for blood flow observation, MI700 might be kept activated continuously and MI800 might be activated in a pulsed fashion. The images derived therefrom contain all necessary information for discriminating between the contributions of ICG and SGM-101.

At least one, in some cases several of the fluorescence images 30.1 through 30.n contain the fluorescence responses from more than one fluorescent dye present in the operating area shown in the images, as explained in the context of FIG. 1. The next step is therefore an image processing step 32 performed on the initially captured white light images 30.0 and fluorescence images 30.1 through 30.n. The white light images 30.0 may be processed according to standard image processing protocols with respect to brightness, contrast and so on, arriving at a processed white light image 34.0, labelled “pWLI” for “processed white light image”.

The two or more fluorescence images 30.1 through 30.n, however, are processed together in image processing step 32, by means of one or more pre-trained artificial intelligence models or simpler mathematical models such as linear combinations of the fluorescence images in order to arrive at processed fluorescence images 34.1 through 34.n, labelled “pFLI.1” through “pFLI.n” in FIG. 3, each containing the local distribution of a single one of the two or more fluorescent dyes used in the operating area. The processing together of the various fluorescence images 30.1 through 30.n taken under different fluorescence excitation illumination modes may be done with in the fluorescence images of a single sequence, or include fluorescence images captured in previous sequences, thus allowing for improved suppression of noise in the fluorescence images. The image processing may include recognizing shifts in the field of view, to which the processing may respond by either reducing the number of sequences from which fluorescence images are used for extracting the individual responses of the various fluorescent dyes, or by applying shifts to the fluorescence images corresponding to the observed shifts in the field of view for the purpose of processing the fluorescent images together.

After the processing 32, the resulting processed white light and fluorescence images 34.0 through 34.n may be input to a post processing step 36. In post processing step 36, it is decided what kind of information is displayed to an operator and how that information is to be displayed. For example, the image 38 displayed to the operator may be one of the pure processed white light image 34.0, a single one or a combination of two or more of the processed separate fluorescence images 34.1 through 34.n of the individual fluorescent dyes, or an overlay of a single one or a combination of two or more of the processed separate fluorescence images 34.1 through 34.n of the individual fluorescent dyes over the processed white light image 34.0.

This process as illustrated in FIG. 3 is repeated for the duration of the procedure, for example a fluorescence guided surgery or the like.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE SIGNS

10 System for multi-dye fluorescence imaging
12 endoscope
14 endoscope shaft
16 camera head
20 controller
22 controller hardware
24.0 white light illumination light source
24.1-24.n fluorescence excitation light sources
30.0 white light image
30.1-30.n fluorescence images
32 image processing
34.0 processed white light image
34.1-34.n images showing local distribution of fluorescent dyes separately
36 post processing
38 resulting image
101 ICG emission spectrum
102 ICG excitation spectrum
103 laser excitation spectrum
104 SGM-101 emission spectrum
105 SGM-101 excitation spectrum
106 laser excitation spectrum

What is claimed is:

1. A method of medical multi-dye fluorescence imaging, the method comprising:
capturing fluorescent images of an operating field in which two or more different fluorescent dyes are present in successive sequences of images with a surgical or diagnostic imaging device, each sequence of images alternating through different modes of excitation lighting adapted for at least one each of two or more of the two or more different dyes, and
processing the captured images of each successive sequence of images in combination with each other, the processing comprising:
determining the respective local distributions of the two or more different fluorescence dyes causing the observed different distributions of brightness in the two or more different fluorescent images of the sequence; and
producing one or more images displaying the determined local distributions of the two or more different fluorescent dyes separately.

2. The method according to claim 1, wherein the processing further comprising inputting the fluorescent images of the sequence of images into at least one artificial intelligence model trained to perform the determination of the respective local distributions of the two or more different fluorescence dyes, or into several artificial intelligence models that have each been trained on at least one of several different dyes.

3. The method according to claim 1, wherein the processing further comprising using linear combinations of the two or more fluorescent images with regard to their respective brightness distributions, the linear combinations being performed with weighting factors determined from known or measured relative fluorescence response strengths of the two or more different fluorescent dyes at the two or more different modes of excitation, the weighting factors chosen such as to separate the responses of the two or more fluorescent dyes from each other.

4. The method according to claim 3, wherein, in each linear combination, the contribution of a specific one of the two or more fluorescent dyes is left, whereas the contributions of the other one or more fluorescent dye or dyes is or are eliminated.

5. The method according to claim 1, wherein each of the different modes of excitation lighting comprises activating a different set of one or more fluorescence excitation lighting sources that are each adapted to produce fluorescence excitation lighting for a different one of the two or more different dyes.

6. The method according to claim 5, wherein at least one of the fluorescence excitation lighting sources is activated or kept activated continuously.

7. The method according to claim 1, further comprising, in each sequence of images, additionally capturing at least one white light image under white light illumination, and producing a composite image of the at least one white light image with an overlay of the determined distribution or distributions of at least one of the two or more different fluorescent dyes.

8. The method according to claim 7, wherein the white light illumination is activated continuously.

9. The method according to claim 5, wherein the overlay is performed using a false color representation having a different color for each of the different fluorescent dyes.

10. A system for medical multi-dye fluorescence imaging, the system comprising:
a controller comprising hardware,
a plurality of light sources controlled by the controller, the plurality of light sources comprising two or more fluorescence excitation illumination light sources configured to provide excitation illumination, wherein each of the two or more fluorescence excitation illumination light sources is configured to generate a different excitation lighting adapted to a different one of two or more different pre-selected fluorescence dyes,
at least one imaging unit comprising a first image sensor sensitive for fluorescence light from the two or more different pre-selected fluorescence dyes,
the controller being configured to:
control the plurality of fluorescence excitation illumination light sources by activating the two or more different fluorescence excitation illumination light sources in successive sequences of activations, each sequence of activations alternating through different modes of excitation lighting adapted for at least one each of two or more of the two or more different dyes,
receive first image data from the first image sensor in synchronization with the successive sequences of activations of the two or more different fluorescence excitation illumination light sources,
process the first image data of each successive sequence of images in combination with each other, the processing comprising determining the respective local distributions of the two or more different fluorescence dyes causing the observed different distributions of brightness in the two or more different fluorescent images of the sequence and
produce one or more images displaying the determined local distributions of the two or more different fluorescent dyes separately.

11. The system according to claim 10, wherein the plurality of light sources further comprising a white illumination light source configured to provide white light illumination, the imaging unit further comprising a second image sensor sensitive in a visible light spectrum,
wherein the controller is further configured to:
control the white illumination light source by activating the white illumination light source such as to, within each successive sequence of activations, alternating through different modes of excitation lighting as well as white light illumination,
receive second image data from the second image sensor in synchronization with the successive activations of the white illumination light source, and
produce a composite image by overlaying the one or more images displaying the determined local distributions of the two or more different fluorescent dyes over a white light illumination image derived from the second image data.

12. The system according to claim 10, wherein the controller is configured to run at least one artificial intelligence model trained to perform the determination of the respective local distributions of the two or more different fluorescence dyes, or into several artificial intelligence models that have each been trained on at least one of several different dyes, to which the fluorescent images of the sequence of images are input.

13. The system according to claim 10, wherein the controller is configured to use linear combinations of the two or more fluorescent images with regard to their respective brightness distributions, the linear combinations being done with weighting factors determined from known or measured relative fluorescence response strengths of the two or more different fluorescent dyes at the two or more different modes of excitation, the weighting factors chosen such as to separate the responses of the two or more fluorescent dyes from each other.

14. Non-transitory computer-readable storage medium storing instructions that cause a computer to at least perform:
capturing fluorescent images of an operating field in which two or more different fluorescent dyes are present in successive sequences of images with a surgical or diagnostic imaging device, each sequence of images alternating through different modes of excitation lighting adapted for at least one each of two or more of the two or more different dyes, and
processing the captured images of each successive sequence of images in combination with each other, the processing comprising:
determining the respective local distributions of the two or more different fluorescence dyes causing the observed different distributions of brightness in the two or more different fluorescent images of the sequence; and
producing one or more images displaying the determined local distributions of the two or more different fluorescent dyes separately.
